(19) United States
(12) Patent Application Publication
(10) Pub. No.: US 2024/0349663 A1
(43) Pub. Date: Oct. 24, 2024

(54) Ganoderma lucidum Designated as Strains M2-100-02 and M2-102-02

(71) Applicant: M2 Ingredients, Inc., Vista, CA (US)

(72) Inventors: Samuel Andrasko, Lakeside, CA (US); Julie Daoust, Vista, CA (US); Steven Lee Farrar, Vista, CA (US)

(21) Appl. No.: 18/641,246

(22) Filed: Apr. 19, 2024

Related U.S. Application Data
(60) Provisional application No. 63/461,528, filed on Apr. 24, 2023.
(60) Provisional application No. 63/636,605, filed on Apr. 19, 2024.

(51) Int. Cl. A01G 18/40; C12N 1/14

(52) U.S. Cl. CPC A01G 18/40; C12N 1/14

(57) ABSTRACT

Exemplary embodiments include Ganoderma lucidum fungus designated as strains M2-100-02 and M2-102-02 as deposited with the ATCC Patent Depository under the Budapest Treaty (10801 University Blvd, Manassas, VA 20110), on Jul. 28, 2023, with the unofficial ATCC Patent Deposit Designation No. PTA-127609 (strain M2-100-02) and the unofficial ATCC Patent Deposit Designation No. PTA-127610 (strain M2-102-02), and the official deposit date of Jul. 28, 2023 and the official Patent Deposit Designation No PTA-127609 (strain M2-100-02) and the official Patent Deposit Designation No. PTA-127610 (strain M2-102-02). In some embodiments, the improved Ganoderma lucidum fungus designated as strains M2-100-02 and M2-102-02 are characterized by having optimized concentrations of bioactive compounds including Ganoderic acids and triterpines. In some embodiments, a Beta-glucan concentration is 20 percent or greater. In some embodiments, Ganoderma lucidum fungus designated as strains M2-100-02 and M2-102-02 are improved to be grown on hydrated hulled oats.

### CROSS REFERENCE TO RELATED APPLICATIONS

This application claims the priority benefit of U.S. Provisional Patent Application Ser. No. 63/461,528 filed on Apr. 24, 2023 and titled “Ganoderma lucidum DESIGNATED AS STRAINS M2-100-02 and M2-102-02.” This application also claims the priority benefit of U.S. Provisional Patent Application Ser. No. 63/636,605 filed on Apr. 19, 2024, and titled “Systems and Methods of Liquid Submerged Fermentation for Expanding Inoculum of Fungal Species.” The aforementioned disclosures are hereby incorporated by reference in their entireties including all patent deposit materials and all references cited therein.

### FIELD OF THE TECHNOLOGY

The present invention belongs to the field of microbial technology. More specifically, Embodiments of the disclosure relate to improved strains of Ganoderma lucidum (commonly known as Reishi).

### BRIEF SUMMARY

Exemplary embodiments include Ganoderma lucidum fungus designated as strains M2-100-02 and M2-102-02 as deposited with the ATCC Patent Depository under the Budapest Treaty (10801 University Blvd, Manassas, VA 20110), on Jul. 28, 2023, with the unofficial ATCC Patent Deposit Designation No. PTA-127609 (strain M2-100-02) and the unofficial ATCC Patent Deposit Designation No. PTA-127610 (strain M2-102-02), and the official deposit date of Jul. 28, 2023 with the official Patent Deposit Designation No. PTA-127609 (strain M2-100-02) and with the official Patent Deposit Designation No. PTA-127610 (strain M2-102-02).

In some aspects, the techniques described herein relate to a Ganoderma lucidum fungus designated as strain M2-100-02 with an unofficial ATCC Patent Deposit Designation No. PTA-127609 (strain M2-100-02) and an official Patent Deposit Designation No. PTA-127609.

In some aspects, the techniques described herein relate to a Ganoderma lucidum fungus designated as strain M2-100-02, characterized by a phenotype having a distributed mass of fruitbodies on top and sides of a substrate.

In some aspects, the techniques described herein relate to a Ganoderma lucidum fungus designated as strain M2-100-02, characterized by a phenotype having fruiting initiation between eighteen to twenty days after inoculation.

In some aspects, the techniques described herein relate to a Ganoderma lucidum fungus designated as strain M2-100-02, characterized by a phenotype optimized to grow on oats.

In some aspects, the techniques described herein relate to a Ganoderma lucidum fungus designated as strain M2-100-02, characterized by a phenotype having a beta-glucan content of 20% to 35%.

In some aspects, the techniques described herein relate to a Ganoderma lucidum fungus designated as strain M2-100-02, characterized by a phenotype having a beta-glucan content of 25% to 35%.

In some aspects, the techniques described herein relate to a Ganoderma lucidum fungus designated as strain M2-100-02, characterized by a phenotype having a beta-glucan content of 20% to 30%.

In some aspects, the techniques described herein relate to a Ganoderma lucidum fungus designated as strain M2-100-02, characterized by a phenotype having a beta-glucan content of 25% to 30%.

In some aspects, the techniques described herein relate to a Ganoderma lucidum fungus designated as strain M2-100-02, characterized by a phenotype having a beta-glucan content of 20% to 25%.

In some aspects, the techniques described herein relate to a Ganoderma lucidum fungus designated as strain M2-100-02, characterized by a phenotype having a beta-glucan content of 25%.

In some aspects, the techniques described herein relate to a Ganoderma lucidum fungus designated as strain M2-100-02, in a powder form.

In some aspects, the techniques described herein relate to a Ganoderma lucidum fungus designated as strain M2-100-02, in a capsule form.

In some aspects, the techniques described herein relate to a Ganoderma lucidum fungus designated as strain M2-100-02, in a hot beverage form.

In some aspects, the techniques described herein relate to a Ganoderma lucidum fungus designated as strain M2-100-02, further including various concentrations of Ganoderic acids and triterpines.

In some aspects, the techniques described herein relate to a method for propagating a Ganoderma lucidum fungus designated as strain M2-100-02, using a liquid spawn, the method including: culturing a mother culture; culturing a production culture, the production culture being a sub-culture of the mother culture; creating a liquid master inoculum using the production culture; diluting the liquid master inoculum to create a diluted liquid master inoculum; creating a biomass product using the diluted liquid master inoculum; slicing the biomass product for dehydration to create a sliced biomass product; dehydrating the sliced biomass product to create a dehydrated sliced biomass product; milling the dehydrated sliced biomass product to select a designated particle size of the dehydrated sliced biomass product; and screening the designated particle size of the dehydrated sliced biomass product.

In some aspects, the techniques described herein relate to a method for propagating a Ganoderma lucidum fungus designated as strain M2-100-02 using a grain spawn, the method including: culturing a mother culture; culturing a production culture, the production culture being a sub-culture of the mother culture; creating a master spawn using the production culture; creating a production spawn using the master spawn; creating a biomass product using the production spawn; slicing the biomass product for dehydration to create a sliced biomass product; dehydrating the sliced biomass product to create a dehydrated sliced biomass product; milling the dehydrated sliced biomass product to select a designated particle size of the dehydrated sliced biomass product; and screening the designated particle size of the dehydrated sliced biomass product.

In some aspects, the techniques described herein relate to a Ganoderma lucidum fungus designated as strain M2-102-02 with an unofficial ATCC Patent Deposit Designation No. PTA-127610 (strain M2-102-02) and an official Patent Deposit Designation No. PTA-127610.

In some aspects, the techniques described herein relate to a Ganoderma lucidum fungus designated as strain M2-102-02, characterized by a phenotype having a distributed mass of fruitbodies on top and sides of a substrate.

In some aspects, the techniques described herein relate to a Ganoderma lucidum fungus designated as strain M2-102-02, characterized by a phenotype having fruiting initiation between eighteen to twenty days after inoculation.

In some aspects, the techniques described herein relate to a Ganoderma lucidum fungus designated as strain M2-102-02, characterized by a phenotype optimized to grow on oats.

In some aspects, the techniques described herein relate to a Ganoderma lucidum fungus designated as strain M2-102-02, characterized by a phenotype having a beta-glucan content of 20% to 35%.

In some aspects, the techniques described herein relate to a Ganoderma lucidum fungus designated as strain M2-102-02, characterized by a phenotype having a beta-glucan content of 25% to 35%.

In some aspects, the techniques described herein relate to a Ganoderma lucidum fungus designated as strain M2-102-02, characterized by a phenotype having a beta-glucan content of 20% to 30%.

In some aspects, the techniques described herein relate to a Ganoderma lucidum fungus designated as strain M2-102-02, characterized by a phenotype having a beta-glucan content of 25% to 30%.

In some aspects, the techniques described herein relate to a Ganoderma lucidum fungus designated as strain M2-102-02, characterized by a phenotype having a beta-glucan content of 20% to 25%.

In some aspects, the techniques described herein relate to a Ganoderma lucidum fungus designated as strain M2-102-02, characterized by a phenotype having a beta-glucan content of 25%.

In some aspects, the techniques described herein relate to a Ganoderma lucidum fungus designated as strain M2-102-02, in a powder form.

In some aspects, the techniques described herein relate to a Ganoderma lucidum fungus designated as strain M2-102-02, in a capsule form.

In some aspects, the techniques described herein relate to a Ganoderma lucidum fungus designated as strain M2-102-02, in a hot beverage form.

In some aspects, the techniques described herein relate to a Ganoderma lucidum fungus designated as strain M2-102-02, further including various concentrations of Ganoderic acids and triterpines.

In some aspects, the techniques described herein relate to a method for propagating a Ganoderma lucidum fungus designated as strain M2-102-02, using a liquid spawn, the method including: culturing a mother culture; culturing a production culture, the production culture being a sub-culture of the mother culture; creating a liquid master inoculum using the production culture; diluting the liquid master inoculum to create a diluted liquid master inoculum; creating a biomass product using the diluted liquid master inoculum; slicing the biomass product for dehydration to create a sliced biomass product; dehydrating the sliced biomass product to create a dehydrated sliced biomass product; milling the dehydrated sliced biomass product to select a designated particle size of the dehydrated sliced biomass product; and screening the designated particle size of the dehydrated sliced biomass product.

In some aspects, the techniques described herein relate to a method for propagating a Ganoderma lucidum fungus designated as strain M2-102-02 for a grain spawn, the method including: culturing a mother culture; culturing a production culture, the production culture being a sub-culture of the mother culture; creating a master spawn using the production culture; creating a production spawn using the master spawn; creating a biomass product using the production spawn; slicing the biomass product for dehydration to create a sliced biomass product; dehydrating the sliced biomass product to create a dehydrated sliced biomass product; milling the dehydrated sliced biomass product to select a designated particle size of the dehydrated sliced biomass product; and screening the designated particle size of the dehydrated sliced biomass product.

### BRIEF DESCRIPTION OF THE FIGURES

The patent or patent application file contains at least one drawing executed in color. Copies of this patent or patent application publication with color drawing(s) will be provided by the Office upon request and payment of the necessary fec. With respect to FIGS. 1-4, the color drawings are necessary as the only practical medium by which aspects of the claimed subject matter may be accurately conveyed. The color of Ganoderma lucidum may vary. Thus, the color drawings help differentiate strains M2-100-02 and M2-102-02 deposited with the ATCC Patent Depository under the Budapest Treaty (10801 University Blvd, Manassas, VA 20110), on Jul. 28, 2023, with the unofficial ATCC Patent Deposit Designation No. PTA-127609 (strain M2-100-02) and the unofficial ATCC Patent Deposit Designation No. PTA-127610 (strain M2-102-02), and the official deposit date of Jul. 28, 2023 with the official Patent Deposit Designation No. PTA-127609 (strain M2-100-02) and with the official Patent Deposit Designation No. PTA-127610 (strain M2-102-02) from other strains of Ganoderma lucidum. The color drawings also help differentiate a stage of growth of strain M2-102-02 and of strain M2-100-02.

FIG. 1 is a color drawing showing morphological characteristics of Ganoderma lucidum fungus designated as strain M2-102-02, according to some embodiments of the present disclosure.

FIG. 2 is another color drawing showing the morphological characteristics of Ganoderma lucidum fungus designated as strain M2-102-02, according to some embodiments of the present disclosure.

FIG. 3 is a color drawing showing the morphological characteristics of Ganoderma lucidum fungus designated as strain M2-102-02 in a bioreactor bag, according to some embodiments of the present disclosure.

FIG. 4 is another color drawing showing the morphological characteristics of Ganoderma lucidum fungus designated as strain M2-102-02 in a bioreactor bag, according to some embodiments of the present disclosure.

FIG. 5 is a flowchart illustrating an exemplary method for propagating a Ganoderma lucidum fungus, according to some embodiments of the present disclosure.

FIG. 6A is a flowchart illustrating a method for propagating a Ganoderma lucidum fungus designated as strains M2-100-02 and M2-102-02 using liquid spawn, according to some embodiments of the present disclosure.

FIG. 6B is a flowchart illustrating a method for propagating a Ganoderma lucidum fungus designated as strains M2-100-02 and M2-102-02 using grain spawn, according to some embodiments of the present disclosure.

### DETAILED DESCRIPTION

Exemplary embodiments include Ganoderma lucidum fungus designated as strains M2-100-02 and M2-102-02 as deposited with the ATCC Patent Depository under the Budapest Treaty (10801 University Blvd, Manassas, VA 20110), on Jul. 28, 2023, with the unofficial ATCC Patent Deposit Designation No. PTA-127609 (strain M2-100-02) and the unofficial ATCC Patent Deposit Designation No. PTA-127610 (strain M2-102-02), and the official deposit date of Jul. 28, 2023 with the official Patent Deposit Designation No. PTA-127609 (strain M2-100-02) and with the official Patent Deposit Designation No. PTA-127610 (strain M2-102-02).

According to some embodiments, Ganoderma lucidum fungus designated as strains M2-100-02 and M2-102-02 have been grown on oats for over twenty years in the laboratory conditions making stains M2-100-02 and M2-102-02 improved strains the are not naturally occurring. For example, Ganoderma lucidum fungus stains M2-100-02 and M2-102-02 have improved characteristics such as being able to be grown on hydrated hulled oats in a laboratory environment unlike wild type strains.

According to some embodiments, Ganoderma lucidum fungus designated as strains M2-100-02 and M2-102-02 are improved, not naturally occurring stains by being able to be grown on oats. For example, the present technology includes cauliflower in agar and liquid media as a source that is high in Hypoxanthine which supports nucleic acid synthesis and energy metabolism in basidiomycetes, which speeds up development of the fungi.

According to some embodiments, Ganoderma lucidum fungus designated as strains M2-100-02 and M2-102-02 are adapted and improved using the following media: Oat agar comprising the following:

| | | | |
|---|---|---|---|
| Agar | 10 | grams | 2.00% |
| Powdered whole oat grain | 7 | grams | 1.40% |
| H20 | 1 | liter | |

According to various embodiments, the above exemplary Oat agar encourages the fungal culture to produce the necessary enzymes needed to grow on oats. The adaption to oats by Ganoderma lucidum fungus designated as strains M2-100-02 and M2-102-02 is a non naturally occurring, learned behavior of strains M2-100-02 and M2-102-02 that allows the fungus to adapt to culturing conditions and prepare it for subsequent culturing on Oats, enabling a fast transition and recovery time during subsequent transfers. For example, strains M2-100-02 and M2-102-02 were grown for a period of 13 days to see a noticeable improvement in the rate of growth on oat media to ensure the necessary amylase enzymes were being produced to encourage the breakdown of starches to sugars to provide the fungus with enough energy to support the growth of the fungus.

In various embodiments, Strains M2-100-02 and M2-102-02 are grown for use in Organic Mushroom (“Om’) products (Reishi Capsules, Reishi powder, Master Blend Powder, Master Blend Capsules, Mushroom Hot Chocolate, Mushroom Coffee, Mushroom Coffee Latte), mushroom gummies and may be sold as a bulk powder under M2 Ingredients, Inc. For example, this fungal species is used in products with immune and sleep benefits including hot beverages and drink mix and protein powders.

Ganoderma lucidum (commonly known as Reishi) is revered in Traditional Medicine as the “Mushroom of Immortality”. For example, Reishi has a variety of attributes. Reishi acts as an immune potentiator and immune modulator including helping to balance and down regulate an overactive immune system. Reishi has also been studied for its cardiovascular health benefits. For instance, Reishi is considered a “superior adaptogen” and Reishi assists users in adapting to mental and physical stressors in various embodiments.

Ganoderma lucidum (commonly known as Reishi), the name being from Greek roots of “brightness, sheen and derma or skin” which describe this shiny woody mushroom. The Chinese name, Ling Zhi, means “herb of spiritual potency” that refers to the adaptogenic effects of this most highly revered, superior tonic. In Traditional Chinese Medicine (TCM), Reishi is known as a “three treasure” herb that harmonizes jing (life force), qi (energy) and shen (spirit). For example, Reishi is often referred to as the “Elixir of Life” and the “Mushroom of Immortality”. Reishi has been well researched for its diverse array of polysaccharides and triterpene compounds. For example, see by Galappaththi et. al. “A Review of Ganoderma Triterpenoids and Their Bioactivities”, which is incorporated by reference for all purposes (See Galappaththi M C A, Patabendige N M, Premarathne B M, Hapuarachchi K K, Tibpromma S, Dai D Q, Suwannarach N, Rapior S, Karunarathna S C. A Review of Ganoderma Triterpenoids and Their Bioactivities. Biomolecules. 2022 Dec. 22; 13 (1): 24. doi: 10.3390/biom13010024. PMID: 36671409; PMCID: PMC9856212). The polysaccharides have been shown to both potentiate and modulate the immune system. For example, acting to support and balance. Research related to triterpene activity has demonstrated adaptogenic effects to support blood pressure management, anti-allergenic properties and mood. Reishi has also been reported to balance endocrine function and hormonal balance.

Exemplary embodiments include ingestible products made from Ganoderma lucidum fungus designated as strains M2-100-02 and M2-102-02, and the products including immune support, cardiovascular support and adaptogenic effects. For example, see Lin Z B. “Cellular and molecular mechanisms of immuno-modulation by Ganoderma lucidum,” which is incorporated by reference for all purposes (See Lin Z B. “Cellular and molecular mechanisms of immuno-modulation by Ganoderma lucidum.” J Pharmacol Sci. 2005 October; 99 (2): 144-53. doi: 10.1254/jphs.crj05008x. PMID: 16230843). For example, showing immune support, the immune-modulating effects of Ganoderma lucidum polysaccharides are extensive, including promoting the function of antigen-presenting cells, mononuclear phygocyte activation, humeral immunity and cellular immunity. A variety of complex polysaccharide compounds in Reishi have been shown to stimulate the immune system by activating immune cells such as macrophages and helper T-cells, as well as increasing immunoglobin levels to produce a heightened response to foreign, non-self cells. (See Chen 2004, EI-Mekkawy 1998, Eo 2000, Lin 2004, Rubel 2010 Zhi 2007), which is incorporated by reference for all purpose. Reishi appears to modulate T-cell activation by acting directly on monocytes. Its polysaccharides have been credited by several researchers with promoting splenic B cell activation and antibody secretion, specifically stimulating tumor necrosis factor (TNF)-alpha and IL-6 production and IFN-gamma release. Reishi is also highly regarded for its cardiotonic properties (See Boh B, Berovic M, Zhang J, Zhi-Bin L. Ganoderma lucidum and its pharmaceutically active compounds. Biotechnol Annu Rev. 2007; 13:265-301. doi: 10.1016/S1387-2656 (07) 13010-6. PMID: 17875480 which is incorporated by reference for all purposes). Researchers have reported that various active compounds in Reishi, especially triterpene compounds, can function to lower blood pressure, lower cholesterol, inhibit atherosclerosis and inhibit platelet aggregation. Reishi has also been reported to improve blood flow and lower oxygen consumption in the cardiac muscle. Reishi also has adaptogenic effects. For example, Reishi is regarded in Traditional Chinese Medicine (TCM) as being the premier adaptogen of the entire Chinese pharmacopeia. Adaptogens arc natural substances that enable our bodies to overcome and adapt to various physical, chemical, and biological stress factors. Reishi mushrooms contain polyphenol antioxidants, polysaccharides, polypeptides, triterpenes and other compounds that through actions known as mediation, down-regulation and up-regulation work together to provide the perfect adaptogenic responses to stress. Reishi, in particular, is highly regarded for support of the entire endocrine system, especially in regards to the adrenal cortex so that the levels of stress hormone cortisol and also adrenalin are kept in balance.

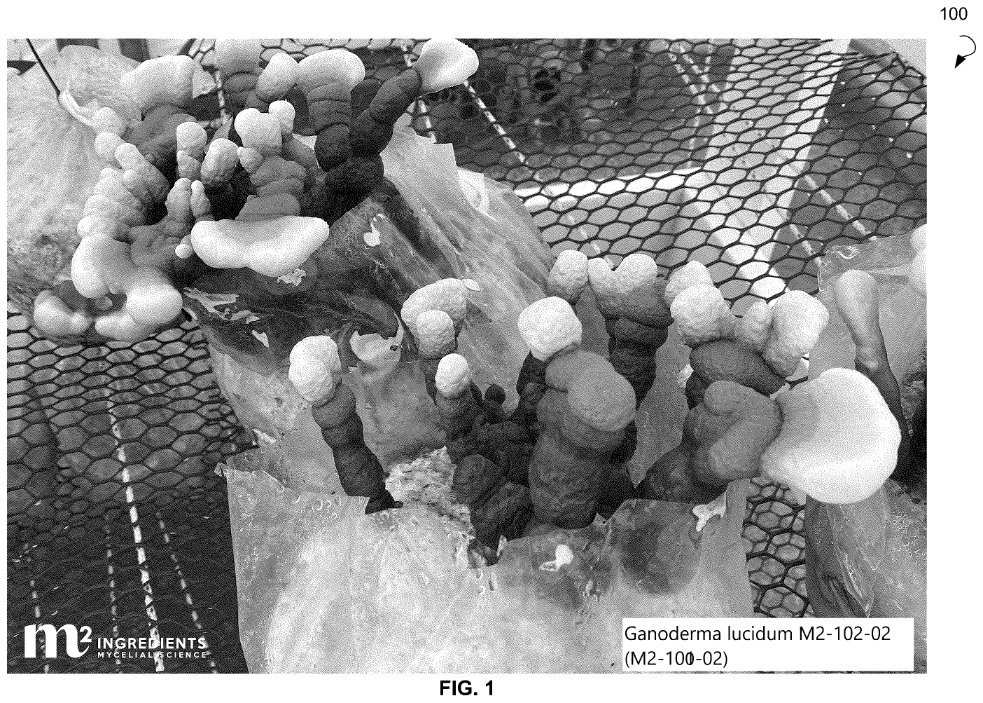

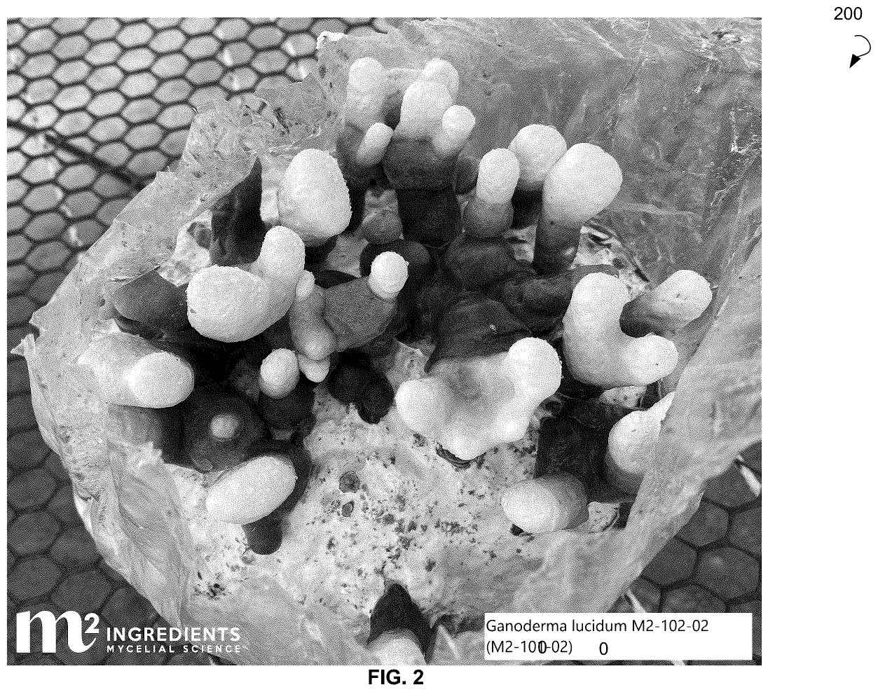

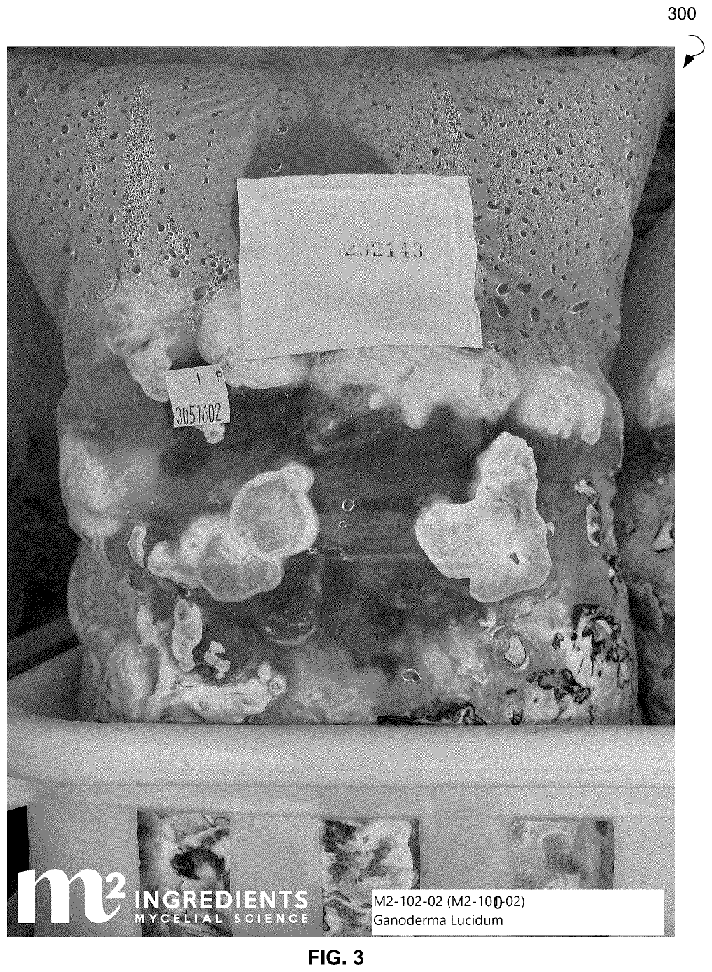

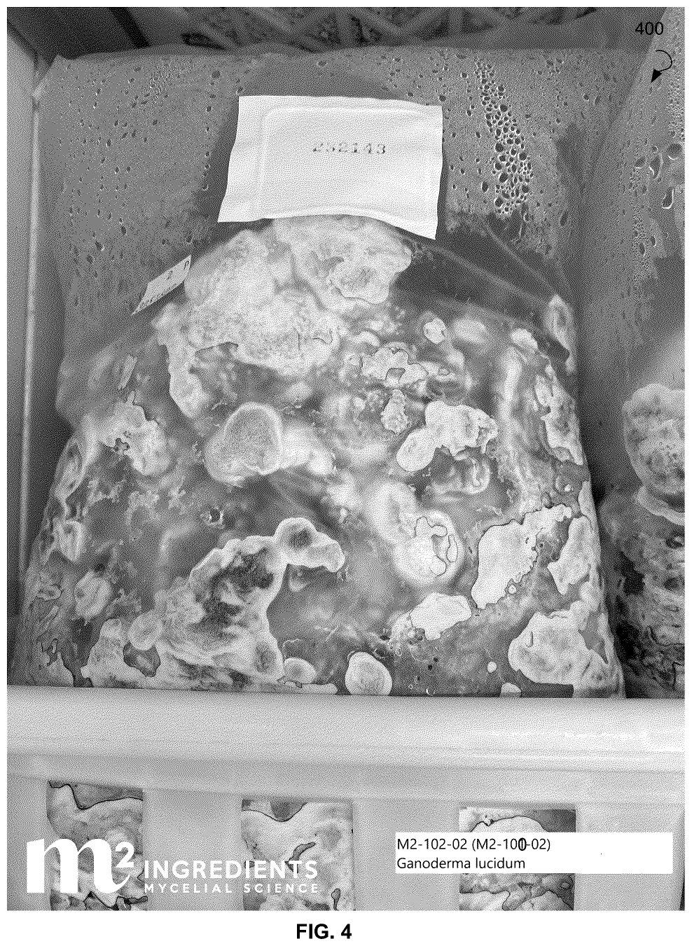

Turning to FIG. 1 through FIG. 4 show FIG. 1 morphological characteristics of Ganoderma lucidum fungus designated as strain M2-102-02 according to various embodiments. FIG. 1 is a color drawing showing morphological characteristics of Ganoderma lucidum fungus designated as strain M2-102-02, according to some embodiments of the present disclosure. FIG. 2 is another color drawing showing the morphological characteristics of Ganoderma lucidum fungus designated as strain M2-102-02, according to some embodiments of the present disclosure. FIG. 2 is a closer view than FIG. 1. FIG. 3 is a color drawing showing the morphological characteristics of Ganoderma lucidum fungus designated as strain M2-102-02 in a bioreactor bag, according to some embodiments of the present disclosure. FIG. 4 is another color drawing showing the morphological characteristics of Ganoderma lucidum fungus designated as strain M2-102-02 in a bioreactor bag, according to some embodiments of the present disclosure. FIG. 4 is a closer view than FIG. 2 of the.

Beta-glucans are polymers of β-D-glucose. They constitute part of cell walls of bacteria and plants, mainly algae and cereals, as well as microscopic fungi including Ganoderma lucidum. Beta-glucans have been studied for their various health benefits, particularly in relation to immune system support and cardiovascular health. Some potential health benefits associated with beta-glucans include immune system support, cardiovascular health, blood sugar control, and weight management, just to name a few. For example, see exemplary Biological Activities of B-Glucan from Ganoderma Species that is described in a recent review by Zhang et. al. “Recent Advances in the Preparation, Structure, and Biological Activities of β-Glucan from Ganoderma Species: A Review,” which is incorporated herein for all purposes (Sec Zhang, Henan, Jingsong Zhang, Yanfang Liu, and Chuanhong Tang. 2023. “Recent Advances in the Preparation, Structure, and Biological Activities of B-Glucan from Ganoderma Species: A Review” Foods 12, no. 15:2975).

In various embodiments, phenotypical characteristics of Ganoderma lucidum designated as strains M2-100-02 and M2-102-02 include the following. Produces a distributed mass of fruitbody on the top and sides of grain substrate from 0.8 to 1 pounds. In some embodiments, Fruits best at 75 degrees Fahrenheit. Beta-glucan content range of 20-35% depending on a number of days of culture before harvest. The specification for this ingredient is set to a minimum of 20% beta glucans and have Ganoderic acids and triterpines at various concentration ranges.

In various embodiments Beta-glucan concentration is 20% or greater and this specific point of at least 20% is critical or special to the operability of strain hot beverages and drinks. Strains M2-100-02 and M2-102-02 with a Beta-glucan concentration of 20% or greater produce a new and unexpected result, which is different in kind and not merely in degree.

In some embodiments a grain spawn with Strains M2-100-02 and M2-102-02 is specifically used for production. For example, a grain spawn is made from sterilized grains that have been inoculated with a live mycelium culture of Strain M2-100-02 or M2-102-02. These inoculated grains are consumed by the growing mass of mycelium of Strains M2-100-02 and M2-102-02. In various embodiments a liquid spawn of strain M2-100-02 or M2-102-02 is used as an alternative method.

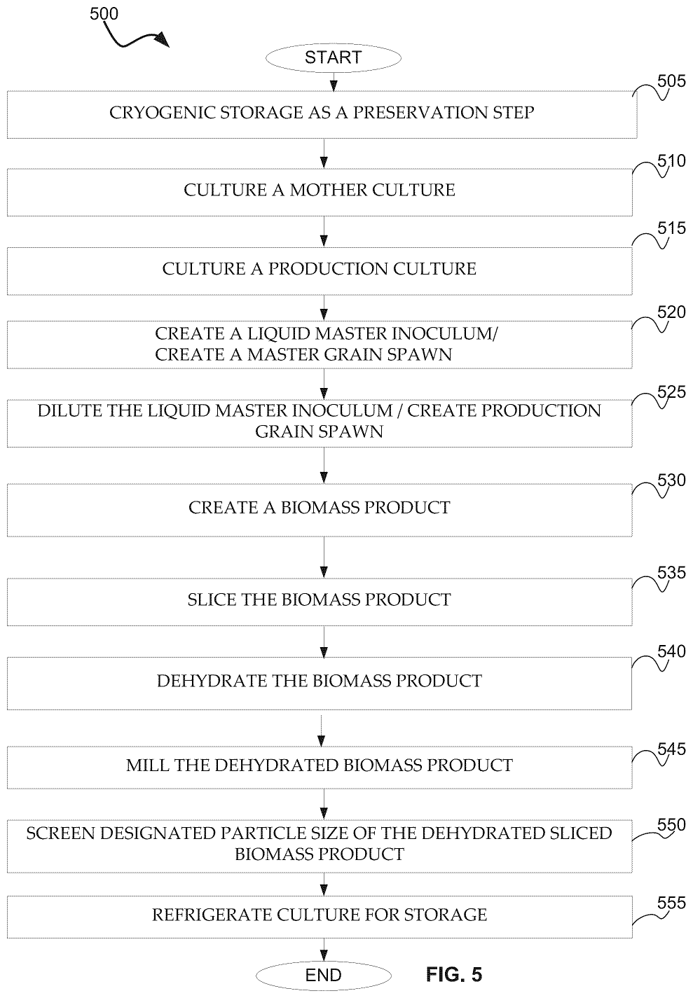

FIG. 5 is a flowchart illustrating an exemplary method for propagating a Ganoderma lucidum fungus, according to some embodiments of the present disclosure. At step 505, cryogenic storage is used as a preservation step. At step 510, the method may include culturing a mother culture. At step 515, the method may include culturing a production culture on a nutrient agar comprised of a mixture of dextrose, potato starch, nutritional yeast and cauliflower powder. The production culture being a sub-culture of the mother culture. At step 520, the method may include creating a liquid master inoculum/create a master grain spawn using the production culture. At step 525, the method may include diluting the liquid master inoculum/create a production grain spawn to create a diluted liquid master inoculum.

In some embodiments, at step 530, the method may include creating a biomass product using the diluted liquid master inoculum. At step 535, the method may include slicing the biomass product for dehydration to create a sliced biomass product. At step 540, the method may include dehydrating the sliced biomass product to create a dehydrated sliced biomass product. At step 545, the method may include milling the dehydrated sliced biomass product to select a designated particle size of the dehydrated sliced biomass product. At step 550, the method may include screening the designated particle size of the dehydrated sliced biomass product. More details according to various embodiments are disclosed below. In various embodiments

In some embodiments, at step 510 the method may include culturing a mother culture. For example, culturing of mother cultures (preserved stock cultures) may be “the first step in the process”. Fresh cultures of the Ganoderma lucidum fungus of the M2-100-02 and M2-102-02 strains are cultured on a nutrient agar comprised a mixture of dextrose, potato starch, nutritional yeast and cauliflower powder and grown for a period of 10-16 days at 75 Fahrenheit until a culture diameter of 30-40 mm is obtained. 5 mm×5 mm square sections of the healthiest part of the culture is cut from the leading edge of growth of the hyphae and placed into a 1.5-2 ml cryogenic storage vial of 10-20 percent glycerol solution and cryogenically stored under Liquid Nitrogen (LN2). For example, a nutrient agar comprised a mixture of dextrose, potato starch, nutritional yeast and cauliflower powder may comprise the following:

| | | | |
|---|---|---|---|
| Dextrose | 10 | grams | 2.00% |
| Potato starch | 2.5 | grams | 0.50% |
| Agar | 10 | grams | 2.00% |
| Nutritional yeast | 5 | grams | 1.00% |
| H20 | 1 | liter | |

According to some embodiments, at the same time 2 mm square sections of the same culture material is transferred to 10-20 culture tubes equal or greater than 25 mm×150 mm in size filled with a nutrient agar comprising a carbon/nitrogen/vitamin/mineral mix and subsequently stored under refrigeration between 34-40 degrees Fahrenheit. For example, a nutrient agar comprising a carbon/nitrogen/vitamin/mineral mix includes the following:

| | | | |
|---|---|---|---|
| Dextrose | 10 | grams | 2.00% |
| Potato starch | 2.5 | grams | 0.50% |
| Agar | 10 | grams | 2.00% |
| Cauliflower | 5 | grams | 1.00% |

According to some embodiments, cultures tubes are sub-cultured every 6-12 months to new culture tubes to verify that the cultures are still active and healthy and to preserve the phenotypic qualities of the original cultures. Cryogenic cultures previously discussed are recovered and sub-cultured into new culture tubes for storage and not used if any unwanted phenotypical variation is observed. Furthermore, only growth representative of the mother culture is used for inoculum.

In some embodiments at 515, the method may include culturing a production culture, the production culture being a sub-culture of the mother culture. For example, culturing of production cultures may be “the second step in the process”. To start the production process, mother cultures are pulled from refrigeration and sub-cultured onto 4 nutrient agar plates. Cultures are allowed to grow for 10-16 days at 75 Fahrenheit until a growth diameter of 30-40 mm is obtained. The healthiest and most representative tissue of the mother culture is sub-cultured into step 520.

In some embodiments at 520, the method may include creating a liquid master inoculum/create a master grain spawn using the production culture. For example, creation of Master spawn also known as “1st generation grain spawn” (G1's) may be “the third step in the process”. An exemplary protocol is as follows: one lb. of hydrated hulled oats, hydrated to a 50-60% moisture content in a water bath at 190-195 Fahrenheit for 30 min to 60 min in a water bath is filled into an appropriate clear autoclavable Bioreactor bag comprised of a polypropylene or high density polyethylene or a Polypropylene/High density polyethylene blend fitted with a microporous filter patch or strip material for gas exchange. The oats are subjected to sterilization in an autoclave for an appropriate time and temperature to fully render any present microorganisms inactive. The bags are cooled in a HEPA filtrated refrigerated cooldown room until the grain is equal to or less than 90 degrees F. Once cooled the bags are delivered to a HEPA filtrated laminar flow hood and a laboratory technician subcultures a 10 mm×10 mm section of the healthiest tissue from step 515 to each individual Master spawn bag. The bag is allowed to incubate at 75-80 degrees Fahrenheit for 11-14 days. During incubation the bag is shaken once a 3 cm colony from around the inoculation site occurs. The bag is left to incubate until the grain is fully consolidated with the mycelium and then shaken again. The bag is then placed under refrigeration at 34-40 degrees Fahrenheit to slow down the metabolism of the culture and stall the growth. The master spawn is then monitored for contamination and regrowth every 3 days until the masters are sent to create production spawn in step 525.

In some embodiments at 525, the method may include diluting the liquid master inoculum/create a production grain spawn to create a diluted liquid master inoculum. For example, see U.S. Provisional Patent Application Ser. No. 63/636,605 filed on Apr. 19, 2024, and titled “Systems and Methods of Liquid Submerged Fermentation for Expanding Inoculum of Fungal Species.” For example, creation of production spawn “second generation spawn” (G2's) may be “the 4th step in the process”. An exemplary protocol is as follows: 7-8.5 pounds of hydrated hulled oats, hydrated to a 40-60% moisture content in a water bath at 190-195 Fahrenheit for 30-60 min is filled into an appropriate clear autoclavable Bioreactor bag comprised of a polypropylene or high-density polyethylene or a Polypropylene/High density polyethylene blend fitted with a microporous filter patch or strip material for gas exchange. The hydrated hulled oats are subjected to sterilization in an autoclave for an appropriate time and temperature to fully render any present microorganisms inactive. The bags are cooled in a HEPA filtrated refrigerated cooldown room until the grain is equal to or less than 90 degrees Fahrenheit. Once cooled the bags are delivered to a HEPA filtrated laminar flow hood and a laboratory technician subcultures the Master grain bags to the production spawn at ratio of 1:10-1:20. The Production spawn bags are then incubated for 12-16 days.

In some embodiments, at step 530, the method may include creating a biomass product using the diluted liquid master inoculum. For example, see U.S. Provisional Patent Application Ser. No. 63/636,605 filed on Apr. 19, 2024, and titled “Systems and Methods of Liquid Submerged Fermentation for Expanding Inoculum of Fungal Species.” For example, Creation of Biomass product may be “the 5th step of the process”. An exemplary protocol is as follows: 600-800 8.5 pounds of hydrated hulled oats, hydrated to a 40-60% moisture content in a water bath at 190-195 Fahrenheit for is filled into an appropriate clear autoclavable Bioreactor bag comprised of a polypropylene or high density polyethylene or a Polypropylene/High density polyethylene blend fitted with a microporous filter patch or strip material for gas exchange. The hydrated hulled oats are subjected to sterilization in an autoclave for an appropriate time and temperature to fully render any present microorganisms inactive. The bags are cooled in a HEPA filtrated refrigerated cooldown room until the grain is equal to or less than 90 degrees Fahrenheit. Once cooled the bags are delivered to a HEPA filtrated laminar flow hood and a laboratory technician subcultures the production spawn to the biomass bags at ratio of 1:180-1:200. The bags are then incubated for 50-70 days at 75-80 Fahrenheit. The bags will contain the fruiting biomass, mycelium, and digested oats.

In some embodiments at 535, the method may include slicing the biomass product for dehydration to create a sliced biomass product. For example, Slicing “may be the 6th step of the process”. For instance, upon harvesting the biomass; the biomass is sliced though a dicer into smaller pieces and placed upon dehydration trays for dehydration.

In some embodiments at 540, the method may include dehydrating the sliced biomass product to create a dehydrated sliced biomass product. For example, dehydration of biomass product may be “the 7th step of the process”. For instance, the trays of biomass product is loaded into a static dehydrator and dried for approximately 24 hours at 180 degrees Fahrenheit and/or into a dynamic continuous convection belt dryer with a range of 195 degrees Fahrenheit to 200 degrees Fahrenheit for approximately 30 to 60 minutes to a moisture content of less than 5%. This process is carefully watched to prevent altering the taste of the final product.

In some embodiments at 545, the method may include milling the dehydrated sliced biomass product to select a designated particle size of the dehydrated sliced biomass product. For example, milling may be “the 8th step of the process”. For instance, the dried biomass is put though a mill and screened to a designated particle size.

In some embodiments at 550, the method may include screening the designated particle size of the dehydrated sliced biomass product. For example, the dried biomass is screened to a designated particle size and bulk packed for customers.

At 555, a culture may be refrigerated for storage.

In some exemplary embodiments, air may be distributed during the incubation process across bags at a rate of 0.7 to 1.2 meters a second.

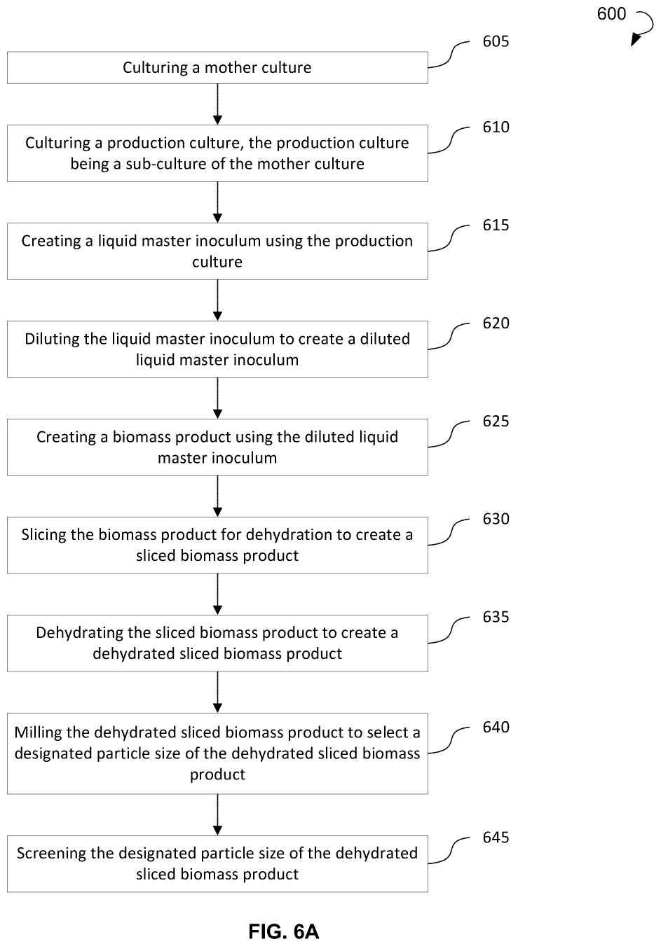

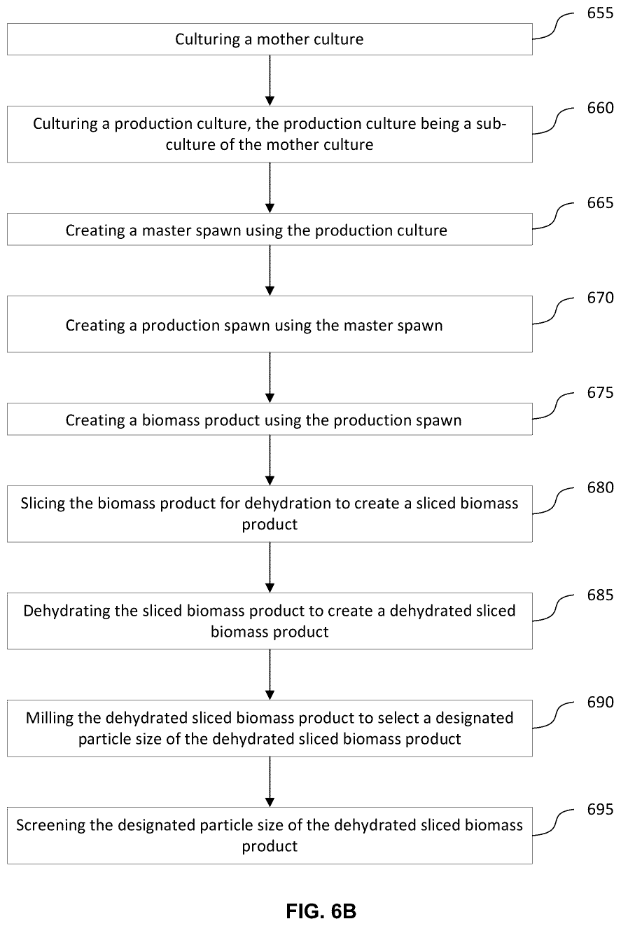

FIG. 6A is a flowchart illustrating a method for propagating a Ganoderma lucidum fungus designated as strains M2-100-02 and M2-102-02 using liquid spawn, according to some embodiments of the present disclosure, the method comprising the following steps. In some embodiments, at step 605, the method may include culturing a mother culture. At step 610, the method may include culturing a production culture, the production culture being a sub-culture of the mother culture. At step 615, the method may include creating a liquid master inoculum using the production culture. At step 620, the method may include diluting the liquid master inoculum to create a diluted liquid master inoculum. In some embodiments, at step 625, the method may include creating a biomass product using the diluted liquid master inoculum. At step 630, the method may include slicing the biomass product for dehydration to create a sliced biomass product. At step 635, the method may include dehydrating the sliced biomass product to create a dehydrated sliced biomass product. At step 640, the method may include milling the dehydrated sliced biomass product to select a designated particle size of the dehydrated sliced biomass product. At step 645, the method may include screening the designated particle size of the dehydrated sliced biomass product. More details according to various embodiments are disclosed herein. For example, a designated particle size may be accomplished by screening the particles using 60 mesh and verification though a particle analyzer.

FIG. 6B is a flowchart illustrating a method for propagating a Ganoderma lucidum fungus designated as strains M2-100-02 and M2-102-02 using grain spawn, according to some embodiments of the present disclosure, the method comprising the following steps. At step 655, culturing a mother culture. At step 660, culturing a production culture, the production culture being a sub-culture of the mother culture. At step 665, creating a master spawn using the production culture. At step 670, creating a production spawn using the master spawn. At step 675, creating a biomass product using the production spawn. At step 680, slicing the biomass product for dehydration to create a sliced biomass product. At step 685, dehydrating the sliced biomass product to create a dehydrated sliced biomass product. At step 690, milling the dehydrated sliced biomass product to select a designated particle size of the dehydrated sliced biomass product. At step 695, screening the designated particle size of the dehydrated sliced biomass product. More details according to various embodiments are disclosed below. In some embodiments, FIG. 6B is a preferred embodiment for propagating the Ganoderma lucidum fungus designated as strains M2-100-02 and M2-102-02 using grain spawn because of high quality product being produced using grain spawn. Additional, details according to various embodiments are disclosed herein.

While various exemplary embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the technology as defined by the appended claims. Thus, the breadth and scope of a preferred embodiment should not be limited by any of the above-described exemplary embodiments.

What is claimed is:

1. A Ganoderma lucidum fungus designated as strain M2-100-02 with an unofficial ATCC Patent Deposit Designation No. PTA-127609 (strain M2-100-02) and an official Patent Deposit Designation No. PTA-127609.

2. The Ganoderma lucidum fungus designated as strain M2-100-02 of claim 1, characterized by a phenotype having a distributed mass of fruitbodies on top and sides of a substrate.

3. The Ganoderma lucidum fungus designated as strain M2-100-02 of claim 1, characterized by a phenotype having fruiting initiation between eighteen to twenty days after inoculation.

4. The Ganoderma lucidum fungus designated as strain M2-100-02 of claim 1, characterized by a phenotype optimized to grow on oats.

5. The Ganoderma lucidum fungus designated as strain M2-100-02 of claim 1, characterized by a phenotype having a beta-glucan content of 20% to 35%.

6. The Ganoderma lucidum fungus designated as strain M2-100-02 of claim 1, characterized by a phenotype having a beta-glucan content of 25% to 35%.

7. The Ganoderma lucidum fungus designated as strain M2-100-02 of claim 1, characterized by a phenotype having a beta-glucan content of 20% to 30%.

8. The Ganoderma lucidum fungus designated as strain M2-100-02 of claim 1, characterized by a phenotype having a beta-glucan content of 25% to 30%.

9. The Ganoderma lucidum fungus designated as strain M2-100-02 of claim 1, characterized by a phenotype having a beta-glucan content of 20% to 25%.

10. The Ganoderma lucidum fungus designated as strain M2-100-02 of claim 1, characterized by a phenotype having a beta-glucan content of 25%.

11. The Ganoderma lucidum fungus designated as strain M2-100-02 of claim 1, in a powder form.

12. The Ganoderma lucidum fungus designated as strain M2-100-02 of claim 1, in a capsule form.

13. The Ganoderma lucidum fungus designated as strain M2-100-02 of claim 1, in a hot beverage form.

14. The Ganoderma lucidum fungus designated as strain M2-100-02 of claim 1, further comprising various concentrations of Ganoderic acids and triterpines.

15. A method for propagating a Ganoderma lucidum fungus designated as strain M2-100-02, using a liquid spawn, the method comprising:
culturing a mother culture;
culturing a production culture, the production culture being a sub-culture of the mother culture;
creating a liquid master inoculum using the production culture;
diluting the liquid master inoculum to create a diluted liquid master inoculum;
creating a biomass product using the diluted liquid master inoculum;
slicing the biomass product for dehydration to create a sliced biomass product;
dehydrating the sliced biomass product to create a dehydrated sliced biomass product;
milling the dehydrated sliced biomass product to select a designated particle size of the dehydrated sliced biomass product; and
screening the designated particle size of the dehydrated sliced biomass product.

16. A method for propagating a Ganoderma lucidum fungus designated as strain M2-100-02 using a grain spawn, the method comprising:
culturing a mother culture;
culturing a production culture, the production culture being a sub-culture of the mother culture;
creating a master spawn using the production culture;
creating a production spawn using the master spawn;
creating a biomass product using the production spawn;
slicing the biomass product for dehydration to create a sliced biomass product;
dehydrating the sliced biomass product to create a dehydrated sliced biomass product;
milling the dehydrated sliced biomass product to select a designated particle size of the dehydrated sliced biomass product; and
screening the designated particle size of the dehydrated sliced biomass product.

17. A Ganoderma lucidum fungus designated as strain M2-102-02 with an unofficial ATCC Patent Deposit Designation No. PTA-127610 (strain M2-102-02) and an official Patent Deposit Designation No. PTA-127610.

18. The Ganoderma lucidum fungus designated as strain M2-102-02 of claim 17, characterized by a phenotype having a distributed mass of fruitbodies on top and sides of a substrate.

19. The Ganoderma lucidum fungus designated as strain M2-102-02 of claim 17, characterized by a phenotype having fruiting initiation between eighteen to twenty days after inoculation.

20. The Ganoderma lucidum fungus designated as strain M2-102-02 of claim 17, characterized by a phenotype optimized to grow on oats.

21. The Ganoderma lucidum fungus designated as strain M2-102-02 of claim 17, characterized by a phenotype having a beta-glucan content of 20% to 35%.

22. The Ganoderma lucidum fungus designated as strain M2-102-02 of claim 17, characterized by a phenotype having a beta-glucan content of 25% to 35%.

23. The Ganoderma lucidum fungus designated as strain M2-102-02 of claim 17, characterized by a phenotype having a beta-glucan content of 20% to 30%.

24. The Ganoderma lucidum fungus designated as strain M2-102-02 of claim 17, characterized by a phenotype having a beta-glucan content of 25% to 30%.

25. The Ganoderma lucidum fungus designated as strain M2-102-02 of claim 17, characterized by a phenotype having a beta-glucan content of 20% to 25%.

26. The Ganoderma lucidum fungus designated as strain M2-102-02 of claim 17, characterized by a phenotype having a beta-glucan content of 25%.

27. The Ganoderma lucidum fungus designated as strain M2-102-02 of claim 17, in a powder form.

28. The Ganoderma lucidum fungus designated as strain M2-102-02 of claim 17, in a capsule form.

29. The Ganoderma lucidum fungus designated as strain M2-102-02 of claim 17, in a hot beverage form.

30. The Ganoderma lucidum fungus designated as strain M2-102-02 of claim 17, further comprising various concentrations of Ganoderic acids and triterpines.

31. A method for propagating a Ganoderma lucidum fungus designated as strain M2-102-02, using a liquid spawn, the method comprising:
culturing a mother culture;
culturing a production culture, the production culture being a sub-culture of the mother culture;
creating a liquid master inoculum using the production culture;
diluting the liquid master inoculum to create a diluted liquid master inoculum;
creating a biomass product using the diluted liquid master inoculum;
slicing the biomass product for dehydration to create a sliced biomass product;
dehydrating the sliced biomass product to create a dehydrated sliced biomass product;
milling the dehydrated sliced biomass product to select a designated particle size of the dehydrated sliced biomass product; and
screening the designated particle size of the dehydrated sliced biomass product.

32. A method for propagating a Ganoderma lucidum fungus designated as strain M2-102-02 for a grain spawn, the method comprising:
culturing a mother culture;
culturing a production culture, the production culture being a sub-culture of the mother culture;
creating a master spawn using the production culture;
creating a production spawn using the master spawn;
creating a biomass product using the production spawn;
slicing the biomass product for dehydration to create a sliced biomass product;
dehydrating the sliced biomass product to create a dehydrated sliced biomass product;
milling the dehydrated sliced biomass product to select a designated particle size of the dehydrated sliced biomass product; and
screening the designated particle size of the dehydrated sliced biomass product.
